(19) United States
(12) Patent Application Publication
(10) Pub. No.: US 2026/0072110 A1
(43) Pub. Date: Mar. 12, 2026

(54) IDENTIFICATION OF TRANSPORT EQUATIONS FROM DYNAMIC MEDICAL IMAGING DATA

(71) Applicants: City of Hope, Duarte, CA (US); Virginia Tech Intellectual Properties, Inc., Blacksburg, VA (US)

(72) Inventors: Ryan Woodall, Duarte, CA (US); Russell Rockne, Duarte, CA (US); Jennifer Munson, Salem, VA (US); Jessica Reynolds, Blacksburg, VA (US)

(21) Appl. No.: 18/872,027

(22) PCT Filed: Jun. 2, 2023
(86) PCT No.: PCT/US2023/024298

§ 371 Date: Dec. 5, 2024

Related U.S. Application Data
(60) Provisional application No. 63/350,300, filed on Jun. 8, 2022.
(60) Provisional application No. 63/429,292, filed on Dec. 1, 2022.

(51) Int. Cl. G01R 33/50; A61B 5/00; A61B 5/055; G06T 7/00; G06T 7/11; G06T 7/246; G16H 20/00; G16H 30/40

(52) U.S. Cl. CPC G01R 33/50; A61B 5/0036; A61B 5/055; G06T 7/0016; G06T 7/11; G06T 7/248; G16H 30/40; G06T 2200/04; G06T 2207/10016; G06T 2207/10081; G06T 2207/10088; G06T 2207/10104; G06T 2207/10108; G06T 2207/10121; G06T 2207/10136; G06T 2207/30024; G06T 2207/30096; G06T 2207/30104; G06T 2207/30241; G16H 20/00

(57) ABSTRACT

Systems and methods are configured to identify underlying governing equations and/or parameters from analyzed medical imaging data and to form a treatment plan for a patient, modify a treatment plan, and/or treat the patient pursuant to a treatment plan. The type of data that is analyzed can vary and is described herein in an example context of identifying equations governing interstitial fluid transport in tissues from contrast-based medical imaging data, including, but not limited to, Gadolinium-based magnetic resonance imaging (MRI), Iodine-based computed tomography (CT), Iodine-based fluoroscopy, radioisotope-based single photon emission computed tomography (SPECT), dynamic contrast-enhanced MRI (DCE-MRI) or radioisotope-based positron emission tomography (PET.)

### CROSS-REFERENCE TO RELATED APPLICATIONS

This application claims priority to U.S. Provisional Application No. 63/350,300, filed on Jun. 8, 2022, and titled “IDENTIFICATION OF TRANSPORT EQUATIONS FROM DYNAMIC MEDICAL IMAGING DATA”, and U.S. Provisional Application No. 63/429,292, filed on Dec. 1, 2022, and titled “IDENTIFICATION OF TRANSPORT EQUATIONS FROM DYNAMIC MEDICAL IMAGING DATA”, the entirety of each of which is incorporated by reference herein.

### STATEMENT AS TO RIGHTS TO INVENTIONS MADE UNDER FEDERALLY SPONSORED RESEARCH AND DEVELOPMENT

This invention was made with government support under Grant Numbers R01 NS115971 and R37 CA222563, awarded by the National Institutes of Health. The government has certain rights in the invention.

### BACKGROUND

Medical imaging is commonly employed to view the human body in order to diagnose, monitor, and/or treat medical conditions. Different imaging modalities provide different types of information about the portion of the body that is imaged. Medical imaging scans can contain a significant amount of information, including dynamic and structural information, which can inform researchers and clinicians of the location of important features, as well as the dynamics of how those features evolve over time. Much of that information is invisible to the naked eye simply by viewing the image or images over time.

### SUMMARY

Disclosed are systems and methods configured to identify underlying governing equations and/or parameters from analyzed medical imaging data. The type of data that is analyzed can vary and is described herein in an example context of identifying equations governing interstitial fluid transport in tissues from contrast-based medical imaging data, including, but not limited to, Gadolinium-based magnetic resonance imaging (MRI), Iodine-based computed tomography (CT), Iodine-based fluoroscopy, radioisotope-based single photon emission computed tomography (SPECT), dynamic contrast-enhanced MRI (DCE-MRI) or radioisotope-based positron emission tomography (PET).

In the example embodiment, the systems and methods are configured to identify transport equations that govern the flow of fluids within tissues in at least 2 or 3 spatial dimensions. The identified equations can then be used for various endeavors, such as to quantify active or passive transport of fluids in tissues, as well as the orientation of flows relative to tumors or other tissue structures. This is in contrast to conventional state of analysis wherein pre-determined pharmacokinetic models are fit on a voxel-by-voxel basis across a single dimension, time, while ignoring transport between voxels. The disclosed systems and methods allow for 1) data-driven discovery of relevant equations (such as fluid transport equations); and 2) spatial analysis of the relevant environment of the data (such as tissue perfusion and interstitial fluid transport on an individual patient and timepoint basis). The disclosed systems and methods may increase the accuracy of previous pharmacokinetic models, which are nested in the more advanced models identified by the disclosed systems and methods. These features can be used to form a treatment plan for a patient, modify a treatment plan, and/or treat the patient pursuant to a treatment plan.

In a non-limiting example method, a contrast agent is administered to bloodstream or tissue of a human or animal subject. Dynamic scan data is collected before, during, and/or after the administration of the contrast agent to the subject. Such scan data is commonly collected and are often acquired in routine clinical care. Interstitial fluid flows and transport through tissue can be important factors for analysis as tissue perfusion is an important factor in progression and response to therapy in multiple disease settings. For example, tumor tissue perfusion has been shown to be predictive of tumor growth, aggression, and response to therapy in individual research projects, quantifying these biomarkers is non-standardized between institutions, and utilizing these pharmacokinetic parameters as predictive biomarkers has not been approved for diagnostic or prognostic use through clinical trials.

Dynamic contrast MRI is a technique for tracking an amount of contrast agent in tissue over time. Typically, this data is analyzed on a pixel-by-pixel basis, allowing quantification of fluid flow (e.g., blood flow) to respective pixel volumes (voxels) and compartmental volume fractions within the voxel (e.g., percent blood volume, percent cell volume, percent matrix volume, etc.) However, as each voxel is analyzed independently, spatial information, such as fluid diffusion and/or velocity in 3 dimensions is not obtained.

Also disclosed are systems and methods for predicting the motion of particles (such as cells or other particles) through a medium (such as tissue) according to a vector field or vector flow field derived from dynamic time series imaging, such as pursuant to the systems and methods described herein. The prediction of such particle motion can be used, for example, to predict the trajectory and/or terminal location (or intermediate location along a pathway) of one particle or a group of particles. In a non-limiting examples, the systems and methods are used to predict a route and/or a terminal location of tumor cell invasion relative to tissue although other uses are within the scope of this disclosure. These features can be used to form a treatment plan for a patient, modify a treatment plan, and/or treat the patient pursuant to a treatment plan

Accordingly, there is an ongoing need for improved systems and methods for modeling/analysis of fluid flow based upon medical imaging data. In general, systems and methods are disclosed herein for mapping dynamic fluid flow based upon imaging data.

In one aspect, there is disclosed a method of mapping dynamic flow, comprising: receiving, by a processor, a plurality of images representing a concentration of a target as a function of position within a tissue, wherein respective images of the plurality of images are acquired at different times; receiving, by the processor, a plurality of candidate functions configured to model the target concentration as a function of time and the weak form of the derivative of the plurality of candidate functions; dividing, by the processor, a region of interest within each of the plurality of images into a plurality of localized regions, including all temporal data; and constructing, by the processor, a vector field visualization of at least one transport parameter of the target wherein the vector field describes weighted weak form derivatives of the candidate functions normalized by the weak time derivatives of the target concentrations within the plurality of localized regions and provides a trajectory and initial velocity for the plurality of localized regions.

In another aspect, there is disclosed a method of mapping dynamic flow, comprising: accessing, by a processor, a vector flow field; defining, by the processor, a trajectory of at least one particle pursuant to the vector flow field; and determining, by the processor, a cumulative sum of terminating particle trajectories for at least one particle, wherein the terminating particle trajectory corresponds to the most likely point of rest for the at least one particle within the vector flow field.

In another aspect, there is disclosed a method of mapping dynamic flow, comprising: delivering a contrast agent into tissue of a subject such that the contrast agent naturally interacts with tissue of the subject; acquiring a time series of images of an interaction of the contrast agent with the tissue; processing data associated with the images pursuant to a weak SINDy process; and generating an image, the image comprising a vector field visualization of at least one transport parameter of the contrast agent through the tissue.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The patent or application file contains at least one drawing executed in color. These and other features will be more readily understood from the following detailed description taken in conjunction with the accompanying drawings, in which:

FIG. 1 is a diagram illustrating one exemplary embodiment of an operating environment including a mapping system configured to map dynamic flow of fluid within a target;

FIG. 2 is a diagram illustrating an embodiment of a general method for modeling dynamic flow;

FIG. 3 is a diagram illustrating an embodiment of a detailed method for modeling dynamic flow;

FIGS. 4A-4H shows sample images for two human patients;

FIG. 5 is a diagram illustrating an embodiment of a method for obtaining a probabilistic prediction of ultimate terminal location(s) of one or more particles flowing through a medium;

FIG. 6 is a diagram illustrating additional details of the method of FIG. 5;

FIGS. 7-9D show sample images related to the methods described herein.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Embodiments of sensing systems and corresponding methods for dynamic analysis of medical image data are discussed herein. It should be appreciated that embodiments of the disclosure are not limited to medical image data and can be employed for analysis of any image data without limit. The disclosed systems and methods can also be used for analysis of other data aside from image data.

As noted above, medical image data contains a large amount dynamic and structural information that allows identification of the location of features within a target (e.g., tissue, blood supply, lymphatic fluid, air) as well as how these features evolve over time. For example, contrast agents are often employed for tracking fluid flow in tissue over time by acquiring a time series of images of the contrast agent and analyzing individual voxels to quantify fluid flow (e.g., blood flow) and volume fractions within the voxel.

In the context of 1D analysis, parameterization of individual voxels involves fitting an ordinary differential equation (ODE) to the data. However, this approach has at least two drawbacks. First, in many cases, ODEs cannot be solved explicitly, and even then, cannot be solved to guarantee a best fit to the data. As such, the solution to the ODE must be approximated. Second, as the solution to the ODE must be approximated, there is no direct way to optimize the ODE solution to the data. Thus, the ODE must be solved iteratively, varying the underlying parameters until a “good enough” fit is found or an arbitrary iteration limit is reached.

Sparse Identification of Nonlinear Dynamics (SINDy) is a recently developed mathematical method used to infer underlying dynamic equations from data, as opposed to relying upon human intuition to understand the underlying variables and their respective relationships. (See Brunton, et al., “Discovering governing equations from data by sparse identification of nonlinear dynamical systems,” Proceedings of the national academy of sciences,” 113, no. 16 (2016), 3932-3937. The entirety of this publication is incorporated by reference). That is, instead of deriving an equation that describes a phenomenon, the relevant equation can be uncovered. Using linear regression, SINDy allows for the discovery of the dynamics of underlying variables to be determined from data. In addition, if a mathematical model is already known to be relevant to a particular dataset, SINDy may be used as a model-inversion method to determine to what degree the assumed model is applicable to the data. In contrast with other existing techniques, SINDy allows for a mathematically “most-likely” solution to be found through simple linear regression directly from the imaging data, which does not require iterative solutions to ODEs, and one singular ODE is solved at the end in order to ensure algorithm success.

As discussed in greater detail below, a SINDy-based approach constitutes a significant improvement over existing methods, as it is faster, more robust to measurement noise (with specialized modifications), and directly uses the measured data instead of a specified model. Notably, models can be limited by an understanding of the underlying physical process of the modeled phenomena, as well as be potentially biased. Additionally, the SINDy-based approach allows examination of higher order processes, leading to a better understanding of the underlying fluid and biological dynamics, and automating the hypothesis generation step for determining the best underlying model.

In the context of 2D analysis, given a dynamically acquired image volume, fluid transport between voxels can be calculated, enabling determination of fluid velocity and diffusivity fields. Diffusion fields are known to be indicators of cellular density and preferential fluid transport direction. Furthermore, advection fields can be important for predicting the distribution of species in tissue (e.g., cells, nutrients, radiotracers, fluids, etc.).

Advection/diffusion models resulting from SINDy-based approaches can enable models to be built that can simulate therapeutic delivery where drugs or cell therapies (e.g., CAR-T cells) go after delivery to brain, blood, or tissue. With an appropriately selected tracer molecule, receptor-ligand dynamics, transport through specific protein channels and pumps, enzymatic or other biological processes can be modeled in vivo and non-invasively. This approach may also be used to predict tumor growth/invasion direction, alerting surgeons to regions where wider tumor margins may be justifiable. Such information can assist clinicians in making informed decisions on which treatment method is mathematically optimal for each individual patient.

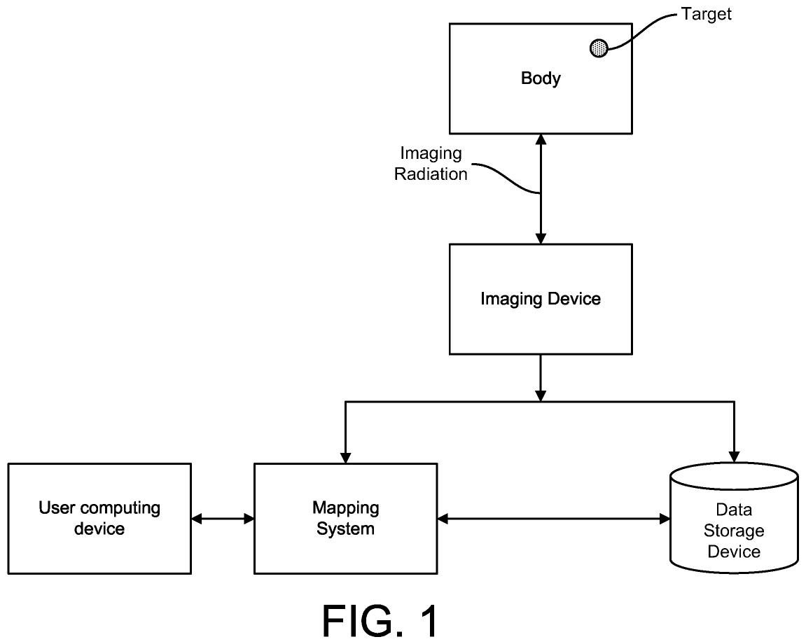

FIG. 1 is a schematic diagram illustrating an embodiment of an operating environment including an imaging device (such as a scanner), a mapping system, a data storage device, and a user computing device including a display device (not shown). The operating environment may also include one or more devices configured to form an image on a medium, such as a printer that prints images on paper. As shown, the imaging device, data storage device, mapping system, and user computing device can be in communication (e.g., via a network).

The imaging device can be configured to acquire one or a plurality of images of a target. In general, the imaging device can include at least one sensor configured to detect an imaging radiation (e.g., reflected from or transmitted through the target). In certain embodiments, the imaging device can include an emitter configured to emit the imaging radiation. In other embodiments, the emitter can be housed separately from the imaging device. Examples of the imaging device can adopt a variety of configurations. Non-limiting examples of the imaging device can include a magnetic resonance imaging (MRI) device, a positron emission tomography (PET) device, a computerized tomography (CT) device, a fluoroscopy device, an ultrasound device, or a single photon emission computed tomography (SPECT) device.

In certain embodiments, the target can be in situ (e.g., within a body such as tissue). In other embodiments, the target can be exogenously administered (e.g., CAR-T cells possessing a reporter gene). Embodiments of the target can adopt a variety of configurations. Non-limiting examples of the target can include at least one of blood, nutrients, cells, proteins, nanoparticles, CAR-T cells and other cell-based therapies, a drug, a tumor, a radiopaque compound, a contrast agent, an antibody, a peptide, an isotope, water or spin-labeled water, glucose, growth factors, or microbubbles. For example purposes, embodiments discussed below assume the target is a contrast agent and the imaging device acquires a plurality of images, each representing concentration of the target as a function of position within the tissue, and acquired at different times (e.g., a time series).

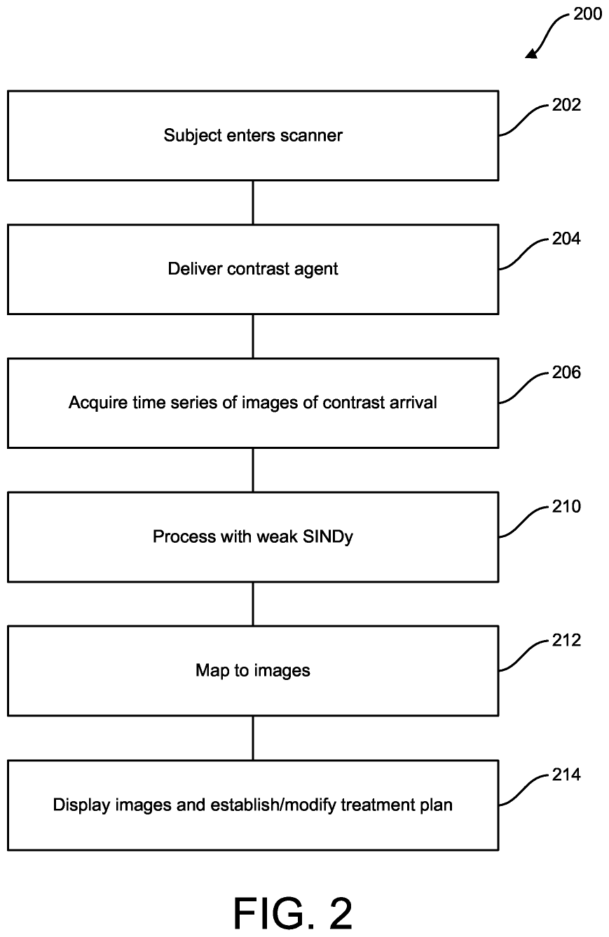

An embodiment of a general method 200 for mapping dynamic flow of the target based upon imaging data acquired by the imaging device is illustrated in FIG. 2. As mentioned, although the method is described in the context of mapping dynamic flow of the target, other variations are within the scope of this disclosure. As shown, the method 200 includes operations 202-214. Embodiments of the method can be implemented by one or more processors of the mapping system and may include greater or fewer operations than illustrated in FIG. 2 and the operations can be performed in an order different than that illustrated in FIG. 2.

In operation 202, a subject (such as a human or animal subject) enters or otherwise couples to the imaging device, which is described in an example context of a scanner. The subject can be positioned in any manner relative to the scanner (either physically or virtually) such that the scanner can obtain one or more images relative to the subject and the target over a period of time. The period of time can be predetermined.

In operation 204, a contrast agent or other directly measurable species is delivered or observed naturally interacting with tissue of the subject. For example, the contract agent can be fluidly injected into the subject. The contrast agent may flow within or otherwise interact with the tissue such as pursuant to a convection-diffusion process. In operation 206, the scanner acquires a time series of images of the interaction of the contrast agent (such as a contrast arrival) with the tissue. The images may be a time series of two dimensional (2D) or three dimensional (3D) images (a 2D or 3D video).

In operation 210, each of a series of images and/or data associated or otherwise descriptive of the images is processed pursuant to a weak SINDy process. Pursuant to this process, a weak form of a partial differential equation (PDE) is utilized. Moreover, one or more test functions are employed, which are smooth functions that preserve original structure of the analyzed data. An integration by parts is used to take derivatives of the test function. The results are then mapped back to the image pursuant to the operation 212.

In operation 214, one or more resulting images and/or data are displayed on a computing device. Such images or data can also be recorded or displayed on a medium, such as by printing on paper for example. Further to operation 214, the images and/or data can also be used to establish and/or modify a treatment plan for the subject such as in response to the steps described herein. The subject may also be treated pursuant to such a treatment plan.

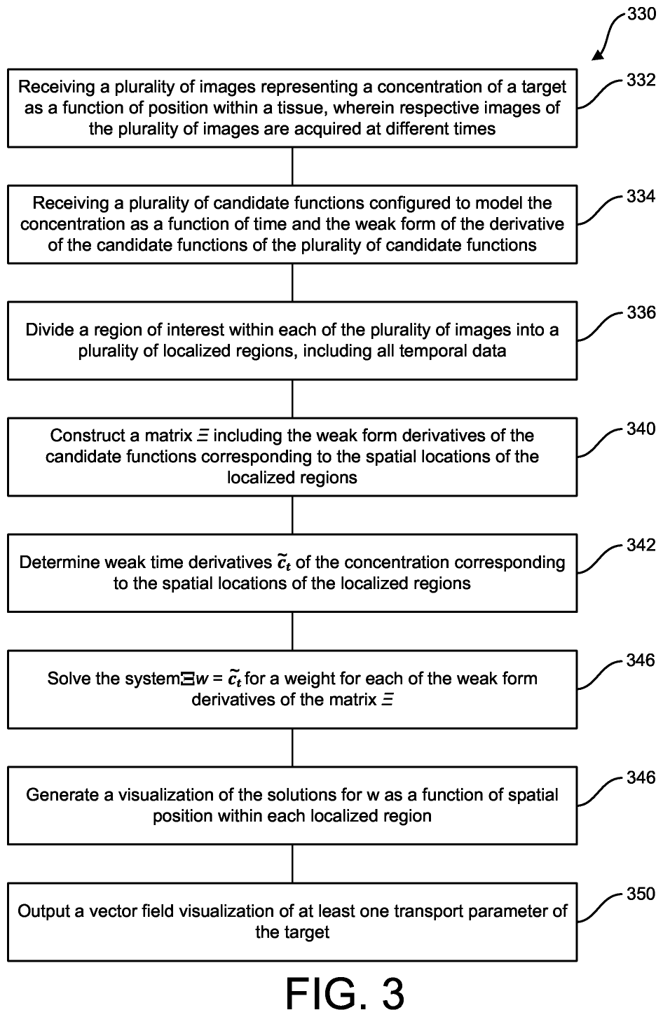

An embodiment of a detailed method 330 for mapping dynamic flow of the target based upon imaging data acquired by the imaging device is illustrated in FIG. 3. As shown, the method 330 includes operations 332-350. Embodiments of the method can be implemented by one or more processors of the mapping system and may include greater or fewer operations than illustrated in FIG. 3 and the operations can be performed in an order different than that illustrated in FIG. 3.

In operation 332, the processor receives a plurality of images representing a concentration c of the target as a function of position within a tissue, c(x, y) in two dimensions or c(x, y, z) in three dimensions. In one example, the plurality of images can be received indirectly from the imaging device after acquisition (e.g., transmitted from the imaging device to the data storage device, and subsequently retrieved by the processor of the mapping system). In another example, the plurality of images can be directly received by the imaging device (e.g., transmitted from the imaging device to the processor of the mapping system.) In a further example, the plurality of images can be received both directly and indirectly by the imaging device.

Respective images of the plurality of images are acquired at different times. The acquisition times can be at approximately equal time intervals. Alternatively, one or more acquisition time intervals between respective images of the plurality of images can be different. The acquisition time interval can be dictated by one or more considerations such as the tissue type, the target type, a type of sensor of the imaging device, the imaging radiation, etc. Accordingly, the plurality of images in a time series can be referred to as c(x, y, t) for data acquired in two dimensions or c(x, y, z, t) for data acquired in three dimensions.

In certain embodiments, post-acquisition processing can be performed to convert as-acquired (raw) image data into concentration data. Such post-acquisition processing can be performed by the imaging device itself, the mapping system, a third computing device or computing cluster, or any combination thereof.

In operation 334, the processor receives at least one candidate function or a plurality of candidate functions that are configured to model the phenomena of interest for the target. Continuing the example above, the phenomena is the target concentration as a function of time. The processor can further receive the “weak form” of the derivative (also referred to merely as “weak derivative”) of the plurality of candidate functions. The purpose of the weak form is to turn the differential equation into an integral equation, which can lessen the computational burden in evaluating derivatives.

In one example, the candidate functions and weak form of the derivative of the plurality of candidate functions can be stored by the data storage device and retrieved by the processor of the mapping system. In another example, the candidate functions and weak form of the derivative of the plurality of candidate functions can be received by the processor via input from a user (e.g., using the user computing device). Combinations of the above may also be performed.

In operation 336, the processor divides a region of interest within each of the plurality of images into a plurality of localized regions, including some or all related temporal data. This localization is a significant contrast with the generalized SINDy framework. That is, SINDy is typically implemented globally when analyzing an image. However, it can be appreciated that when analyzing flow of the target (e.g., a contrast agent) within the body (e.g., tissue), a single equation or set of equations may fail to adequately capture the underlying phenomena dictating the manner of fluid flow due to tissue heterogeneities. That is, a variety of differences can exist between different regions of a tissue (e.g., density, tissue composition, etc.) Thus, in order to properly generate maps of dynamic fluid flow, respective localized regions are analyzed separately to identify the candidate function most suitable to describe fluid flow within the localized regions.

In one embodiment, the localized regions can be a predetermined number of pixels a and b in two dimensions (a×b) or a predetermined number of pixels a, b, c in 3 dimensions (a×b×c). In certain embodiments, each of the localized regions can have the same side length (e.g., square in two dimensions or cube in three dimensions). In other embodiments, the size of respective localized regions can differ from one another. The size or methodology for delineating individual regions of interest may vary, based on the type of medical imaging used (e.g., CT vs MRI vs PET), the spatial and temporal resolution of the imaging modality, the scale of the dynamics under investigation (e.g., local voxel vs. whole-organ, changing dynamics in time), the subject (e.g., Animal or human), organ under investigation (e.g., brain vs. liver), signal-to-noise ratio of the imaging data, and the desired local coupling and smoothness of the output. A separate embodiment of these localized regions could be a plurality of global multi-dimensional Gaussian kernels, with means centered at the x, y (and z-, for 3D) coordinates of each pixel, where the entirety of the image is used to perform the weak SINDy model regression, but is weighted strongest at the space and time corresponding to the location of the mean of the Gaussian kernel employed.

In operation 340, a matrix 8 is constructed of candidate functions and the weak form of derivatives of the candidate functions corresponding to the spatial locations of the localized regions. As a non-limiting example, the candidate functions can be advection-diffusion source equation for fluid flow, or may contain other terms, including higher order terms, or sinusoidal/exponential terms. A non-limiting example of an advection-diffusion source equation for transport of metabolite with underlying fluid motion is the partial differential equation (PDE) illustrated in Equation 1 below:

$$\frac{\partial c ( x , y , t )}{\partial t} = ∇ \cdot ( D ( x , y ) ∇ c ) - ∇ \cdot \vec{u} ( x , y ) c + R \\ where \frac{\partial c ( x , y , t )}{\partial t} \qquad ( Eq . 1 )$$

represents the rate of change of the contrast, ∇·(D(x, y)∇c) represents diffusion of the contrast agent (e.g., motion between voxels due to random motion), ∇·\vec{u}(x, y)c represents advection of the contrast agent (e.g., motion following bulk fluid flow between voxels), and R represents the internal source of the contrast agent (e.g., how contrast enters/leaves the voxel internally). R is given by Equation 2 below:

$$R = {K}^{trans} ( x , y ) \cdot ( {F}_{AIF} - {v}_{e}^{- 1} c ( x , y ) ) + {v}_{p} \frac{\partial {F}_{AIF}}{\partial t} \qquad ( Eq . 2 )$$

where:
K^{trans}(x, y) corresponds to a rate of fluid transfer in a voxel

$$( {F}_{AIF} - {v}_{e}^{- 1} c ( x , y ) )$$

corresponds to a difference between blood concentration and tissue concentration; and

$${v}_{p} \frac{\partial {F}_{AIF}}{\partial t}$$

corresponds to the change of concentration in blood times blood volume fraction

In operation 342, weak time derivatives of the concentration corresponding to the spatial locations of the localized regions are determined. Each derivative is convolved with a test function, such as a known polynomial test function, with known and efficiently determined derivatives Ψ from the family:

$${Ψ}_{x} = {{C}_{1} ( 1 - a x )}^{p} {( b x - 1 )}^{q} \\ Ψ = < {Ψ}_{x} , {Ψ}_{y} , {Ψ}_{t} >$$

Such that

$${\int }_{0}^{T} {\int }_{{x}_{0}}^{{x}_{F}} {\int }_{{y}_{0}}^{{y}_{F}} Ψ \frac{\partial c}{\partial t} dydxdt = \text{} - {\int }_{0}^{T} {\int }_{{x}_{0}}^{{x}_{F}} {\int }_{{y}_{0}}^{{y}_{F}} \frac{\partial Ψ}{\partial t} c ( x , y , t ) dydxdt \approx {\tilde{c}}_{t} \qquad ( Eq . 3 )$$

This process removes the dependency on numerical differentiation of the potentially noisy state data c(x, y, t), while maintaining the structure of the regression problem and projecting into a smooth polynomial function space. Note that because the test functions and all derivatives are 0 at the boundaries, the boundary terms vanish, simplifying the corresponding calculation.

In operation 344, the system is solved for a weight of the weak form derivatives of the matrix. This system may then be solved for w, using either a sparse solver such as Lasso, or simple pseudo-inverse. These methods are equivalent to reconstructing the vector ct from all variables listed in the library through nested linear regression using the weights w.

In operation 346, a visualization of the solutions is generated for the matrix as a function of spatial position within each of the localized regions. In operation 350, a vector field visualization is output for at least one transport parameter of the target. The particular transport parameter that is used can vary. For example, the transport parameter may be at least one of velocity, diffusion, permeability, and hydraulic conductivity.

In addition, as described above with respect to the operations of FIG. 2, images and/or data can be displayed on a display device and/or printed on paper. The resulting images and/or data can be used to establish and/or modify a treatment plan and a subject can be treated pursuant to such a treatment plan.

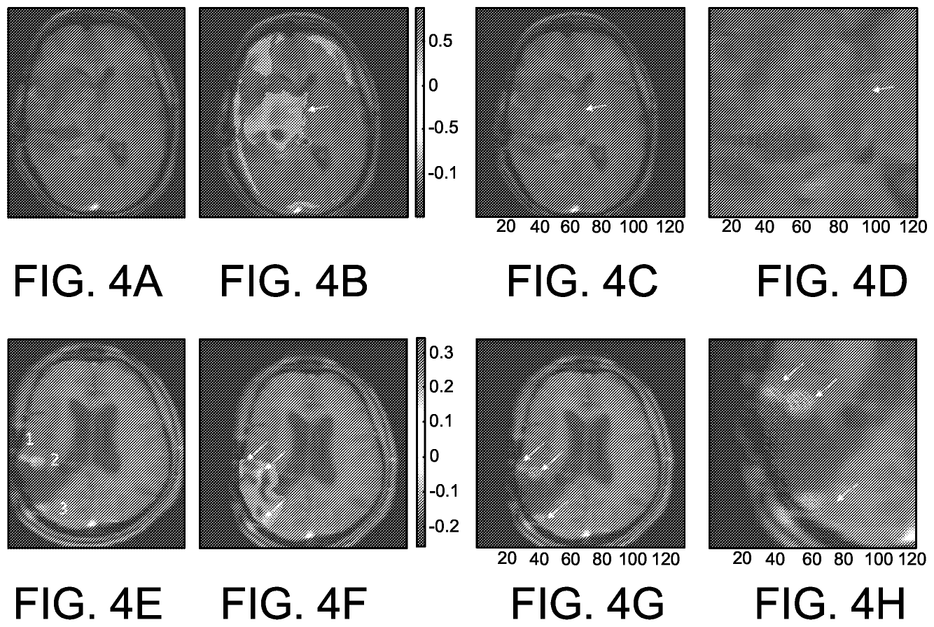

FIG. 4A-4H shows sample images for two human patients including a first patient (labeled FIGS. 4A-4D) and a second patient (labeled FIGS. 4E-4H). At least some of the images were generated pursuant to the systems and methods described herein. The images shown in FIG. 4A-4H are MRI images related to brain tumors and specifically MRI images related to a post gadolinium contrast enhanced T1-weighted MRI of recurrent glioblastoma. It should be appreciated that the systems and methods described herein are not limited to such MRI/brain tumor scenarios and can be used for other scenarios.

The images 4A-4D relate to the first patient and show a brain tumor at least partially due to recurrent glioblastoma. The image 4B shows increased perfusion of contrast due to compromised blood brain barrier extending from the primary tumor mass across the corpus collosum into the contralateral hemisphere. At the same spatial location, there is shown lateral advective flow from the left hemisphere to the right hemisphere. Images 4C and 4D show this advection labeled as an arrow of fluid velocity originating in the left hemisphere towards the right hemisphere (as shown in image 4C and with additional detail in image 4D). Increased perfusion (Ktrans) in the corpus collosum indicates presence of tumor due to breakdown of the blood brain barrier, while the advective field identified using the weak SINDY processes (as described herein) in the same region indicates additional potential for malignant tumor cell invasion across hemispheres. Using prior methods, advective transport across the corpus collosum leading to tumor invasion in the contralateral hemisphere would not be attainable. The information provided by the systems and methods disclosed herein can be used to inform a surgical or other therapeutic plan such as radiation to include this invasive region, thus preventing or delaying spread of the tumor into the right hemisphere.

FIG. 4E-4H show the second patient. FIG. 4E shows 3 distinct recurrent glioblastoma lesions post-resection. Analysis of model terms identified by the weak SINDY methods identifies fundamental differences between the otherwise indistinguishable lesions. Lesions 1 and 2 are vascularized, but lesion 1 has more perfusion than lesion 2, suggesting that lesion 1 may be more sensitive to radiotherapy as it is better oxygenated due to blood leakage as shown in FIG. 4F. Moreover, analysis of terms identified by weak SINDY indicate that lesions 1 and 2 leak fluid into the resection cavity, while lesion 3 leaks fluid into surrounding tissue, as shown with arrows in FIG. 4G and with additional detail in FIG. 4H. Advective transport identified by weak SINDY indicates that lesion 3 has strong potential to invade into surrounding tumor, while the flow patterns around lesions 1 and 2 may limit invasion of the tumor into surrounding tissue. These patient and lesion-specific flow characteristics are not available from prior methods which rely on pre-defined equations that are not individualized. These physics-based findings may allow individualization of treatment to specific lesions according to 1) risk of disease progression, 2) potential to respond to perfusion limiting therapy and 3) guide targeted therapy based on flow and transport characteristics.

Now disclosed are systems and methods for predicting the motion of particles (such as cells or other particles) through a medium (such as tissue) according to a vector field or vector flow field derived from dynamic time series imaging. Such vector flow fields can be derived pursuant to the systems and methods described above. The prediction of such particle motion can be used, for example, to predict the trajectory and/or terminal location (or intermediate location along a pathway) of one particle or a group of particles. In a non-limiting examples, the systems and methods are used to predict a route and/or a terminal location of tumor cell invasion relative to tissue. For example, brain tumor cells invade along nutrient gradients and preferentially follow interstitial flow (flow in space outside of cells in tissue or stroma) when invading healthy brain tissue. As such, given the underlying field of fluid flow within the stroma, preferential routes of tumor cell invasion within the brain may be calculated and predicted. The SINDy-related methods described above may be used to discover, estimate and/or output the interstitial velocity field in 2 or 3 dimensions within a tumor in vivo. The methods described below further use differential equations to integrate such output over the provided velocity fields, and predict or identify 1) the routes by which tumor cells may invade into neighboring tissue, and 2) regions of neighboring tissue that are at high risk for tumor invasion, including but not limited to, recurrence after surgical tumor resection.

Current MRI processing methods only obtain local perfusion kinetic parameters (Ktrans, Kep, Vp), and do not consider transport of contrast agent between pixels. The methods described below are particularly suited for the SINDy method described herein to provide a velocity field, so that it may analyze the velocity field and predict the location where tumor cells are likely to invade. From this velocity field, the methods predict potential trajectories by which tumor may invade, and provide regions of high likelihood of invasion from the aggregated trajectories.

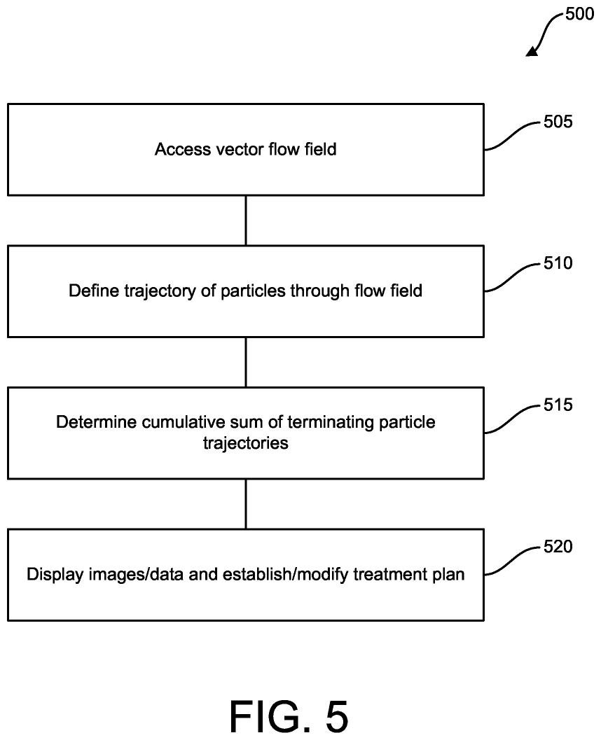

FIG. 5 illustrates an embodiment of a method 500 for obtaining a probabilistic prediction of ultimate terminal location(s) of one or more particles (such as one or more cells or other particles) based upon one or more previously obtained vector fields. In an example embodiment, the vector field are acquired by the previously discussed methods of FIGS. 2 and 3 although they can also be acquired pursuant to other methods. The resulting prediction of terminal location(s) can be used in a variety of scenarios such as, for example, to predict the ultimate tissue location of drug delivered via a cannula or to predict the spread of metastatic cancer cells or radioactive particles. Other scenarios include for example catheter placement, brachytherapy, and CAR-T cell tracking, and drug infusion. Embodiments of the method can be implemented by one or more processors of the mapping system and may include greater or fewer operations than illustrated in FIG. 5 and the operations can be performed in an order different than that illustrated in FIG. 5.

In operation 505, a vector flow field is accessed or obtained. The vector flow field can be one or more equations that predict the motion or other behavior of particles through a medium, such as cells through tissue. For example, the vector flow field may be one or more equations that define velocity and/or position of the particles. In an example, a velocity field \vec{u}(x, y, z) is obtained or provided wherein particles in the velocity field travel along its streamlines. As mentioned, such vector flow field may be obtained or acquired as a result of the previously discussed methods of FIGS. 2 and 3 (or other methods).

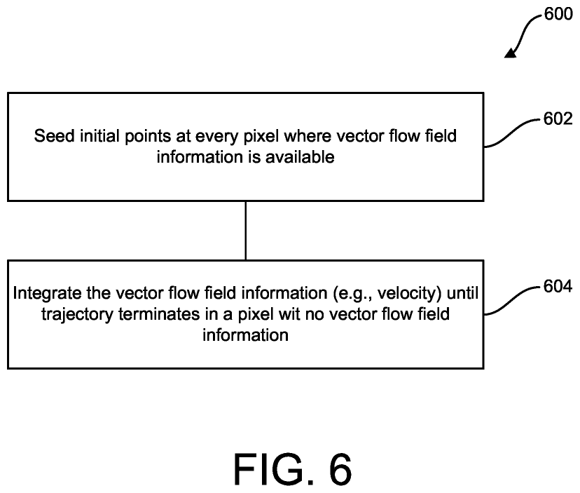

In operation 510, a trajectory or trajectories of the particles flowing through the flow field and pursuant to the vector flow field is defined, as discussed in more detail below with reference to the operations of FIG. 6. The trajectories can be used to define the motion of the particles and the ultimate terminal locations of one or more particles or groups of particles. A trajectory can be an integral curve or path whose tangent vectors coincide with the vector field. Given an initial particle position \vec{s}(t=0)=<x₀, y₀, z_{σ}>=sxî+syĵ+sz\hat{k} in space, and initial particle velocity \vec{u}_{particle}(t=0)=\vec{u}(x₀, y₀, z₀), a particle (e.g., cellular) trajectory s_{particle}(t) may be calculated using differential equations. It is known that velocity (\vec{u}_{particle}) is the first time-derivative of position (s_{particle}):

$${\partial }_{t} {\vec{s}}_{particle} ( t ) = {\vec{u}}_{particle} ( t ) . \qquad ( Eq . 4 )$$

The Fundamental Theorem of Calculus is used to cancel out the derivative on the left-hand side of Equation 4 through integration, thus recovering the position of the particle as a function of time by integrating both sides of the equation with respect to time:

$${\int }_{0}^{t} {\partial }_{τ} {\vec{s}}_{particle} ( τ ) \partial τ = {\vec{s}}_{particle} ( t ) - {\vec{s}}_{particle} ( 0 ) = {\int }_{0}^{t} \vec{u} ( τ ) \partial τ , \qquad ( Eq . 5 )$$

where τ is a dummy variable of integration. Because the dimensions x, y, z (and their corresponding unit vectors î, ĵ, \hat{k}) are orthogonal, they may be separated and integrated independently. The known quantity, \vec{s}_{particle}(0) is then shifted to the right-hand side to achieve the following system of three equations (referred to as Equation 6).

$${\vec{s}}_{particle} ( t ) = {s}_{x} ( t ) \hat{ι} + {s}_{y} ( t ) \hat{J} + {s}_{z} ( t ) \hat{k} = \text{} … \hat{ι} {\int }_{0}^{t} {u}_{x} ( τ ) \partial τ + \hat{J} {\int }_{0}^{t} {u}_{y} ( τ ) \partial τ + \hat{k} {\int }_{0}^{t} {u}_{z} ( τ ) \partial τ + {\vec{s}}_{particle} ( 0 )$$

The system of Equation 6 may be solved pursuant to a variety of methods including for example using computer tools such as ODE45 in MATLAB. As discussed further below with reference to FIG. 6, the particle trajectories are initially seeded at particular locations such as pixels.

In operation 515, the defined trajectories are used to determine a cumulative sum of the terminating trajectories for the one or more particles. In other words, a final position (or an intermediate position) of the particles or groups of particles is determined based upon the previously obtained trajectories. This identifies where the particles that are traveling through the flow field will accumulate. In the context of an image or group of images, this may be done on a predetermined unit basis, such as on a pixel-by-pixel basis. In the context of the particles being cancer cells, this process predicts, for example, where cancer cells leave a tumor bulk and invade into the surrounding normal tissue.

In operation 520, one or more resulting images and/or data are displayed on a computing device. Such images or data can also be recorded or displayed on a medium, such as by printing on paper for example. The images and/or data can also be used to establish and/or modify a treatment plan for the subject such as in response to the steps described herein. The subject may also be treated pursuant to such a treatment plan.

As discussed, the method of FIG. 5 includes an operation 510 of defining one or more trajectories of particles through the flow field. FIG. 6 illustrates an embodiment of a method 600 for defining such trajectories in the context of the trajectories being mapped to an image formed of pixels or other units.

In operation 602, for every pixel (or other unit) for which the vector information is available (such as velocity information), an initial point of each particle is seeded with such vector information. In the example context of analyzing flow of a particle i relative to a tumor, a tumor volume is segmented into units such as voxels. The method places a particle i at the center of each voxel contained within the tumor. In an embodiment, the location of the center of each voxel serves as the initial point or position \vec{s}ᵢ(0) of particle i, although the initial point within the voxel can vary.

In operation 604, the vector flow field information is integrated over the velocity flow field until the trajectory terminates in a pixel with no velocity information. This results in the direction and speed of the trajectory for each particle as well as the pixel, voxel (or other predetermined unit) for which the particle terminates. Pursuant to the tumor example, for each of i=1, 2, . . . , N of N voxels in the tumor, the velocity field as obtained by the methods of FIGS. 2 and 3 described above (or other velocity determination algorithm) are integrated in relative time (pursuant to Equation 6). This results in N individual trajectories in the original image space. Upon finishing the integration for each N particles over relative time t_{final}, the positions of each trajectory over time are displayed or recorded and binned according to the containing imaging voxel. The number of trajectories in an individual voxel is proportional to the number of tumor cells which are predicted to invade this location. This provides a physics- and biology-informed prediction of the location of tumor invasion or recurrence after resection surgery.

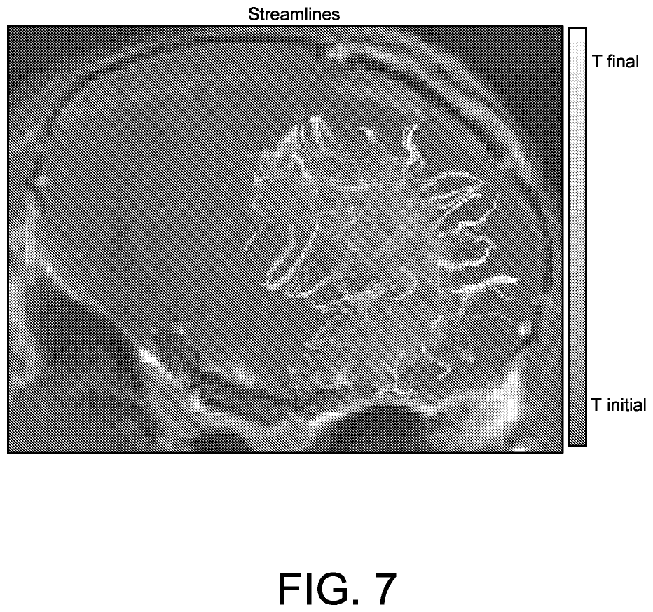

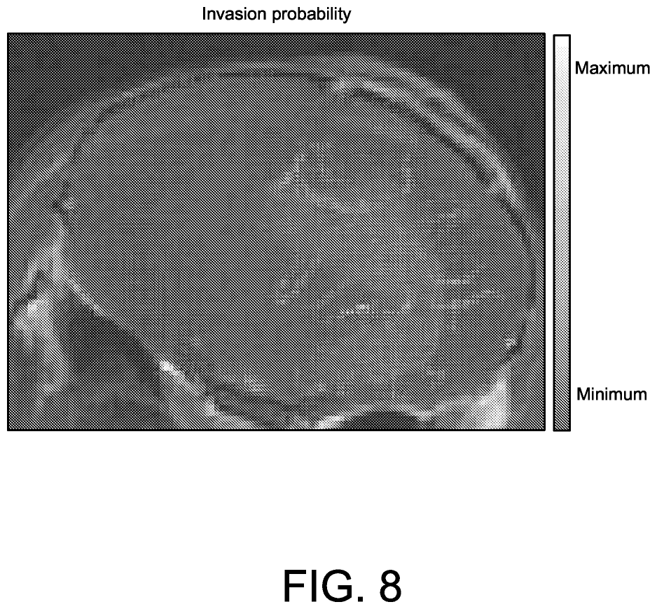

FIG. 7 shows a sample image of a murine glioblastoma tumor region in 2D with particle trajectories also shown in 2D wherein the particle trajectories originate within a murine glioblastoma tumor region in 2D. FIG. 7 indicates time changes by gradient shade, from the initial time T=0 in to a final time T final. Some trajectory routes co-locate, which indicates likely routes of tumor invasion. FIG. 8 shows the sum total number of trajectories traversing through each voxel that originate within the tumor bulk. FIG. 8 indicates few trajectories through this voxel, and also indicates a high number of trajectories, which is then converted to a probability pursuant the methods described herein. For example, certain voxels in this image represent over 100 trajectories crossed through these voxels, indicating a high likelihood of invading tumor cells indicative of progression or recurrence at this location.

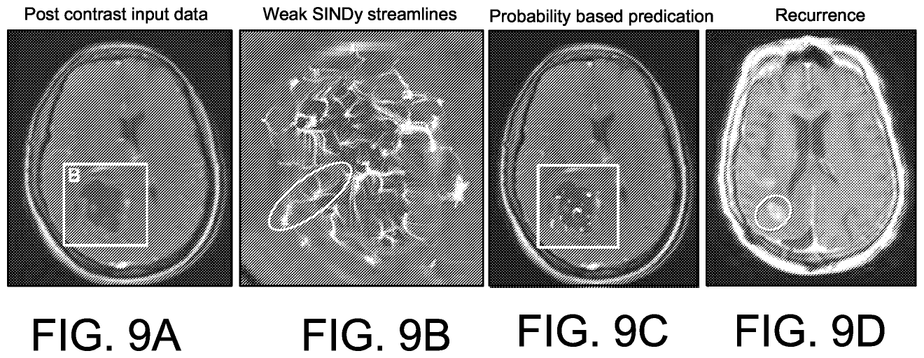

FIGS. 9A-9D illustrate a case study in the application of the disclosed methods to human data. The progression of figures illustrates the prediction of tumor recurrence post-resection (data from the Ivy-Gap Glioblastoma Multiforme (GBM) database). FIG. 9A show post gadolinium contrast T1-weighted MRI image of a brain including a glioblastoma tumor. FIG. 9B shows an enlarged or zoomed-in image of the tumor region in FIG. 9A, with streamlines of interstitial flow. The circle in FIG. 9B indicates an organized flow region, similar to a drainage basin where all flowing fluid converges to a single point. FIG. 9C shows all identified points of convergence. One high probability convergence point that is outside of the tumor boundary is shown in FIG. 9C. FIGS. 9B and 9C indicate a high-probability region of recurrence external to the original tumor. FIG. 9D depicts images of the same patient 8-months after initial tumor is resected, with a recurrent lesion, which was correctly predicted pursuant to the systems and methods described herein.

Where the disclosed systems and methods are used to select the size of the ‘test functions’ in the context of dynamic contrast imaging, it is beneficial to have a delineation between “signal” (the true amount of contrast measured) and “noise” (random perturbations due to uncontrollable fluctuations in the MRI acquisition). The disclosed methods can be affected where the noise is greater than 10% of the maximum true signal. In cases of large noise, the test functions selected may be very large, preventing the detection of smaller fluctuations in perfusion and transport parameters wherein the smaller fluctuations are within a predetermined range of fluctuation values. It can be beneficial for there to be least 10 samples in each direction (x, y, z, and time) to be analyzed, or at least 12 samples. The method removes edge-elements from the analysis, due to “Gibbs phenomenon” when convolving the data with test functions.

The disclosed method can faithfully operate on a true map of “concentration” of the quantity of interest. For MRI, it can be desirable to have an acquisition of a T₁₀ map prior to dynamic contrast imaging. For CT or radionuclide imaging (PET, SPECT), attenuation correction may be desirable.

The disclosed systems and methods were tested and validated as described in the non-limiting examples below.

### Example 1—in Silico

The method was tested in silico in 2D on simulated data, with known velocity fields and diffusion parameters. The simulations included 0.1% noise (as a percent of maximum signal) added for stabilization of the parameterization method. The simulations included divergent flow from a central source and Poiseuille flow through a pipe (zero-slip boundaries.) The method accurately recovered diffusion and velocity values when there was no spatial change in diffusion. Pursuant to the in silico process, all parameters were perturbed by noise, and sharp changes were smoothed out by the smoothness of the test functions. The method accurately recovered direction and magnitude of fluid flow when diffusion was spatially constant. In a situation where diffusion was not spatially constant, the change in diffusion may be mixed with the velocity to produce an “apparent velocity” parameter.

The method was also tested in silico on standard perfusion parameters wherein the method recovered the Ktrans parameter with relative accuracy.

### Example 2—In Vitro

The method was tested in 3D using a small carrier device that holds a porous hydrogel above a collection well. The carrier device was used to force contrast agent through the hydrogel by an above-positioned pressure-head. That is, the contrast agent was first placed atop a porous hydrogel, and then a pressure-head was applied above a gadolinium bolus, forcing the bolus through the hydrogel into a collection well underneath.

A 3D imaging phantom was constructed to observe the bolus of contrast agent travel into porous hydrogel under the gravity-driven pressure-head. The phantom was imaged at 7T using DCE-MRI, with an isotropic voxel resolution of 0.2 mm, and an imaging resolution of 30 s per frame. The bolus of gadolinium then flowed through the phantom, and preferentially leaked through the right of the phantom, where the hydrogel was less cross-linked. The fastest flow of the contrast agent was observed above the hydrogel, where there was no resistance to flow. The flow then slowed within the hydrogel, due to decreased hydraulic conductivity. Finally, the flow accelerated as it leaked through a region of lower crosslinking, into the collection well underneath the phantom. The observed results were consistent with the expected flow profile of a contrast agent flowing through a porous medium, under the laws of Stokes (laminar) flow, and Darcy flow through porous medium. The measured magnitude of the contrast agent wavefront was consistent with the estimated flow-field.

The method recovered higher velocity at regions above the hydrogel, and slower velocities within the hydrogel. The method arrived at a mean velocity that was highly accurate (1.18 um/s), when compared to visually measuring the apparent velocity of the contrast agent (1.26 um/s). The results demonstrate the ability of the disclosed method to accurately estimate apparent fluid velocity and contrast agent diffusivity. The method was able to detect regions of inhomogeneity in the medium corresponding to inhomogeneities in the parametric maps that would otherwise be missed with a wavefront approach.

### Example 3—In Vivo Mouse

An in vivo mouse study captured differences in tumor-associated interstitial flow in differing tumor placements. The method was used to estimate fluid velocity in a tumor of a mouse. A comparison was performed between parametric output from model regression method and immune-histochemistry as a test of the method's ability to detect perfusion in the tumor. ξₜᵣₐₙₛ is a measure, in vivo, of how leaky the vasculature is. In histology, Evans blue is delivered to the blood, and leaks into the surrounding tissue. Evans blue and ξₜᵣₐₙₛ are highly (qualitatively) correlated in position within the tumor. ξ_{VIF} iis a measure of blood vessel presence in vivo, and CD31 is a stain which stains for vaculature. Locations of bright CD31 are also expressed near regions of high vasculature shown by ξ_{VIF} in vivo.

### Example 4—Examination of the Contrast Transport Dynamics in Glioblastoma Multiforme and Primary Breast Cancer

Results of the methodology were performed retrospectively on patient data. The data suggested that on average, contrast agent has an apparent velocity of 102 um/s in glioblastoma, and 18.8 um/s in breast cancer. This data suggests that there are clinical differences between cancers of different organs from a fluid-transport perspective. This evidences that these measurements may be useful to clinicians in developing individualized treatment plans for individuals.

The subject matter described herein can be implemented in analog electronic circuitry, digital electronic circuitry, and/or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

1. A method of mapping dynamic flow, comprising:
receiving, by a processor, a plurality of images representing a concentration of a target as a function of position within a tissue, wherein respective images of the plurality of images are acquired at different times;
receiving, by the processor, a plurality of candidate functions configured to model the target concentration as a function of time and the weak form of the derivative of the plurality of candidate functions;
dividing, by the processor, a region of interest within each of the plurality of images into a plurality of localized regions, including all temporal data; and
constructing, by the processor, a vector field visualization of at least one transport parameter of the target wherein the vector field describes weighted weak form derivatives of the candidate functions normalized by the weak time derivatives of the target concentrations within the plurality of localized regions and provides a trajectory and initial velocity for the plurality of localized regions.

2. The method of claim 1, wherein the candidate functions are partial derivatives of the data, used as terms in weak partial differential equations.

3. The method of claim 1, wherein the candidate functions include at least the vascular input function (VIF) and its time derivative.

4. The method of claim 3, wherein the candidate functions further include at least one of an advection function, a diffusion function, and a perfusion function.

5. The method of claim 2, wherein the imaging device is a magnetic resonance imaging (MRI) device, a positron emission tomography (PET) device, a computerized tomography (CT) device, a fluoroscopy device, an ultrasound device, or a single photon emission computed tomography (SPECT) device.

6. The method of claim 1, wherein at least one transport parameter of the target is velocity, diffusion, permeability, hydraulic conductivity, effective diffusivity, Peclet number, divergence, curl, and volumetric flux.

7. The method of claim 1, wherein the target is at least one of blood, nutrients, cells, proteins, nanoparticles, CAR-T cells and other cell-based therapies, a drug, a tumor, a radiopaque compound, a contrast agent, an antibody, a peptide, an isotope, water or spin-labeled water, glucose, growth factors, or microbubbles.

8. The method of claim 1, further comprising receiving the plurality of images comprises:
acquiring, by an imaging device, the plurality of images by an imaging device; and
transmitting, by the imaging device, the acquired plurality of images to the processor.

9. The method of claim 1, further comprising receiving the plurality of images comprises:
receiving, by a data storage device, the plurality of images acquired by an imaging device; and
transmitting, by the data storage device, the acquired plurality of images to the processor.

10. The method of claim 1, further comprising establishing a treatment plan pursuant to the vector field visualization of the transport parameter.

11. The method of claim 10, further comprising modifying the treatment plan pursuant a result of an analysis of the vector field visualization.

12. The method of claim 10, further comprising treating a patient pursuant to the treatment plan.

13. The method of claim 1, wherein the data structure comprises a matrix Ξ and further comprising:
constructing, by the processor, the matrix Ξ including the weak form derivatives of the candidate functions corresponding to the spatial locations of the localized regions;
determining by the processor, weak time derivatives \tilde{c}ₜ of the concentration corresponding to the spatial locations of the localized regions;
solving, by the processor, the system Ξw=\tilde{c}ₜ for w, where w is a weight for each of the weak form derivatives of the matrix Ξ; and
generating, by the processor, a visualization of the solutions for the system as a function of spatial position within each localized region.

14. The method of claim 1, wherein the candidate functions are selected to be large perfusion functions to prevent detection of smaller fluctuations in perfusion parameters, wherein the smaller fluctuations are within a predetermined range of fluctuation values.

15. The method of claim 2, wherein the imaging device is a magnetic resonance imaging (MRI) device and further comprising acquiring a T₁₀ map.

16. A method of mapping dynamic flow, comprising:
accessing, by a processor, a vector flow field;
defining, by the processor, a trajectory of at least one particle pursuant to the vector flow field; and
determining, by the processor, a cumulative sum of terminating particle trajectories for at least one particle
wherein the terminating particle trajectory corresponds to the most likely point of rest for the at least one particle within the vector flow field.

17. The method of claim 16, wherein the vector flow field is obtained pursuant to the method of claim 1.

18. The method of claim 16, wherein the vector flow field relates to velocity of the at least one particle.

19. The method of claim 16, wherein the vector flow field comprises at least one equation that predicts a motion of particles through a medium.

20. The method of claim 16, wherein the at least one particle is at least one of a cancer cell, CAR-T cell, and a radioactive particle.

21. The method of claim 16, wherein defining, by the processor, a trajectory of at least one particle pursuant to the vector flow field comprises seeding, by a processor, an initial point of the at least one particle.

22. The method of claim 21, wherein the initial point comprises a pixel of a display.

23. The method of claim 16, further comprising displaying at least one terminating particle trajectory on a computer display.

24. The method of claim 16, further comprising establishing a treatment plan pursuant to the method of claim 16.

25. The method of claim 24, wherein the treatment plan comprises at least one of catheter placement, brachytherapy, CAR-T cell tracking, and drug infusion.

26. A method of mapping dynamic flow, comprising:
delivering a contrast agent into tissue of a subject such that the contrast agent naturally interacts with tissue of the subject;
acquiring a time series of images of an interaction of the contrast agent with the tissue;
processing data associated with the images pursuant to a weak SINDy process; and
generating an image, the image comprising a vector field visualization of at least one transport parameter of the contrast agent through the tissue.

27. The method of claim 26, wherein the transport parameter comprises velocity, diffusion, permeability, hydraulic conductivity, effective diffusivity, Peclet number, divergence, curl, and volumetric flux.

28. The method of claim 26, further comprising coupling the subject to the scanner such that the scanner obtains one or more images relative to the subject over a period of time.

29. The method of claim 26, wherein time series of images comprises at least one of a time series of two dimensional (2D) images and three dimensional (3D) images.
